(19) United States
(12) Patent Application Publication
(10) Pub. No.: US 2007/0218556 A1
(43) Pub. Date: Sep. 20, 2007

(54) Measuring nutrients in plants and soils by laser induced breakdown spectroscopy

(76) Inventors: Ronny D. Harris, Los Alamos, NM (US); Pat J. Unkefer, Los Alamos, NM (US); Michael H. Ebinger, Santa Fe, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(21) Appl. No.: 11/640,612

(22) Filed: Dec. 18, 2006

Related U.S. Application Data
(60) Provisional application No. 60/751,584, filed on Dec. 16, 2005.

(51) Int. Cl. G01N 33/24

(52) U.S. Cl. 436/25

(57) ABSTRACT

A process for analyzing the nutrient status of plant matter and/or soil for one or more nutrients selected from among calcium, potassium, nitrogen, sulfur, phosphorus, magnesium, chlorine, iron, boron, manganese, zinc, copper, nickel and molybdenum is described and includes contacting said plant matter and/or soil with a laser source capable of inducing breakdown of the sample whereby an emission from said sample occurs; and, analyzing said spectral emission for determination of an amount of said one or more nutrients. A process for analyzing the heavy metal content of plant matter and/or soil, or of fertilizers or soil amendments is also described.

### RELATED APPLICATIONS

This application claims the benefit of U.S. provisional application Ser. No. 60/751,584 filed Dec. 16, 2005

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates generally to nutrient analysis methods from soil and/or plant matter, more particularly, to nutrient analysis systems using laser-induced breakdown spectroscopy.

### BACKGROUND OF THE INVENTION

Laser-induced breakdown spectroscopy has been demonstrated to be an effective tool in analysis of total soil carbon measurements (see, Ebinger et al., Soil Sci. Soc. Am. J., vol. 67, pp. 1616-1619, 2003). Analysis of soils and plant matter is of critical importance to modern agriculture. Present analytical techniques generally require tedious extraction techniques prior to analysis by atomic absorption spectroscopy or by a calorimetric technique.

A need remains for an analytical technique that eliminates the need for preliminary extraction processes. It is desirable that such an analytical technique can provide results for a variety of targeted species without the need for a wide range of wet chemistry, calorimetric or chromatographic analysis techniques.

### SUMMARY OF THE INVENTION

In accordance with the purposes of the present invention, as embodied and broadly described herein, the present invention includes process for analyzing the nutrient status of plant matter and/or soil for one or more nutrients selected from among calcium, potassium, nitrogen, sulfur, phosphorus, magnesium, chlorine, iron, boron, manganese, zinc, copper, nickel and molybdenum including contacting said plant matter and/or soil with a laser source capable of inducing breakdown of the sample whereby an emission from said sample occurs, and, analyzing said spectral emission for determination of an amount of said one or more nutrients.

The present invention further provides a process for analyzing plant matter and/or soil for one or more heavy metals selected from among iron, lead, arsenic, chromium, and cadmium including contacting said plant matter and/or soil with a laser source capable of inducing breakdown of the sample whereby an emission from said sample occurs; and, analyzing said spectral emission for determination of an amount of said one or more heavy metals.

The present invention further provides a process for analyzing a fertilizer or a soil amendment for one or more heavy metals selected from among iron, lead, arsenic, chromium, and cadmium including contacting said fertilizer or soil amendment with a laser source capable of inducing breakdown of the sample whereby an emission from said sample occurs; and, analyzing said spectral emission for determination of an amount of said one or more heavy metals.

### BRIEF DESCRIPTION OF THE DRAWINGS

FIG. 1 shows a schematic drawing of a LIBS instrument including micro-plasma collection, detection and spectral resolution of a sample.

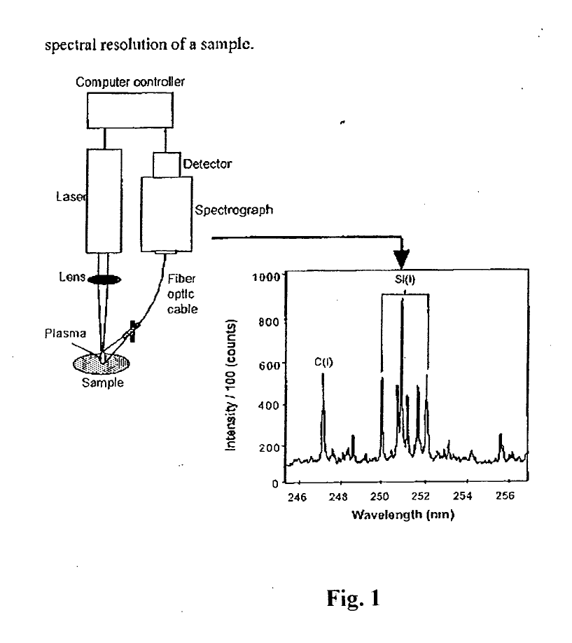

FIG. 2 shows a calibration curve for iron in plant leaves with a limit of detection (LOD) of about 35 ppm, the determination at 239.56 nm wavelength for the iron.

FIG. 3 shows a calibration curve for barium in plant leaves with a limit of detection (LOD) of about 35 ppm, the determination at 493.41 nm wavelength for the barium.

FIG. 4 shows a calibration curve for calcium in plant leaves with a limit of detection (LOD) of about 650 ppm, the determination at 854.21 nm wavelength for the calcium.

FIG. 5 shows a calibration curve for magnesium in plant leaves with a limit of detection (LOD) of about 330 ppm, the determination at 280.27 nm wavelength for the magnesium.

FIG. 6 shows a calibration curve for sodium in plant leaves with a limit of detection (LOD) of about 45 ppm, the determination at 588.99 nm wavelength for the sodium.

FIG. 7 shows a calibration curve for strontium in plant leaves with a limit of detection (LOD) of about 7 ppm, the determination at 421.55 nm wavelength for the strontium.

FIG. 8 shows a calibration curve for potassium in plant leaves with a limit of detection (LOD) of about 975 ppm, the determination at 766.49 nm wavelength for the potassium.

FIG. 9 shows a table containing the various selected wavelengths in nanometers for the targeted species and the LOD for those species.

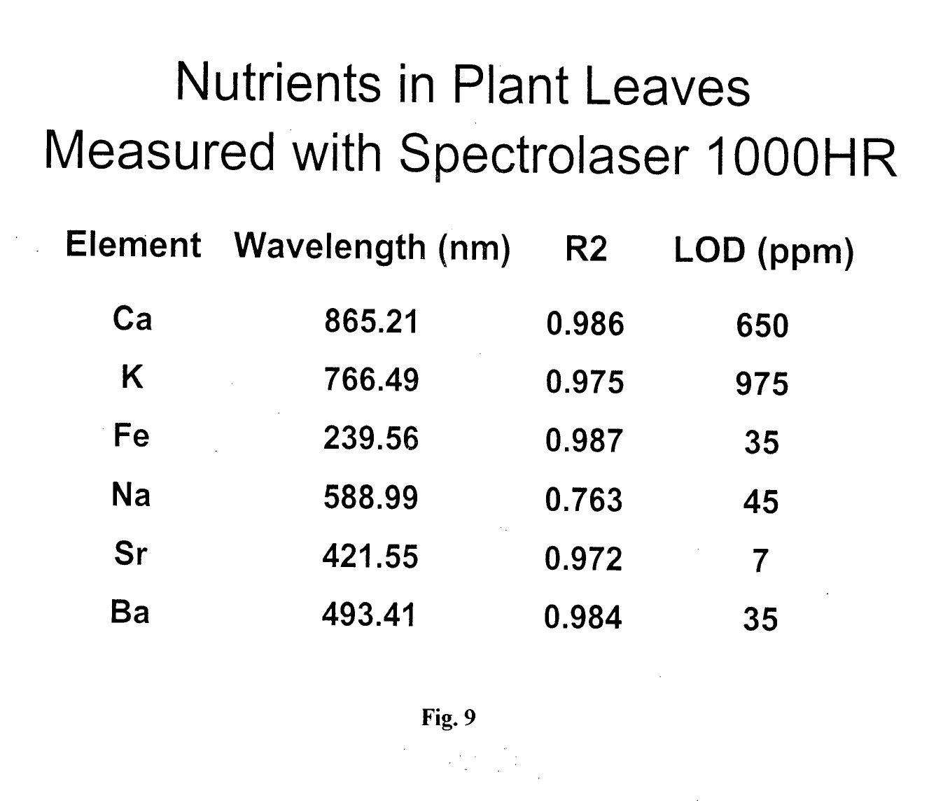

FIG. 10 shows a calibration curve for phosphorus in a spiked soil sample with a limit of detection (LOD) of about 2000 ppm, the determination at 253.56 nm and 255.32 nm wavelengths for the phosphorus.

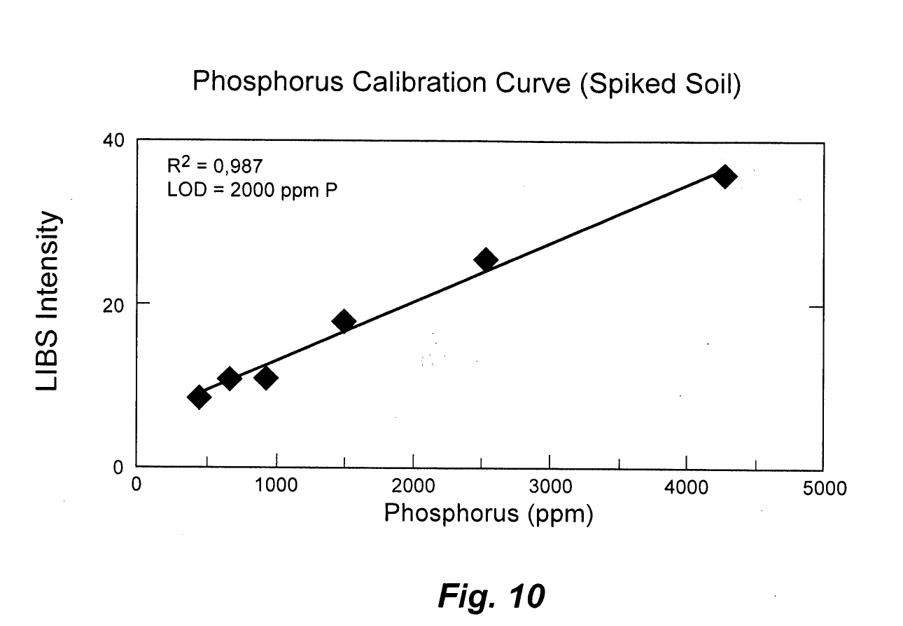

FIG. 11 shows a calibration curve for nitrogen in a spiked soil sample (a sand/clay mixture) with a limit of detection (LOD) of about 0.3 percent nitrogen at 0.04 Torr or 0.1 percent nitrogen under argon, the determination at 742.36 nm, 744.23 nm and 746.83 nm wavelengths for the nitrogen.

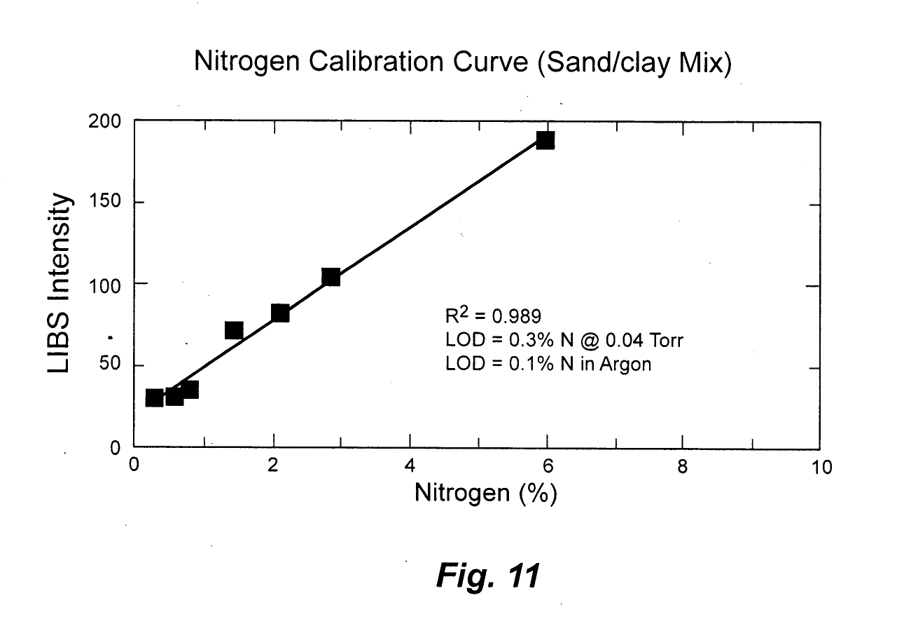

FIG. 12 shows a calibration curve for sulfur in a spiked soil sample (a sand/clay mixture) with a limit of detection (LOD) of about 0.3 percent sulfur at 7 Torr, the determination at 545.38 nm and 564.00 nm wavelengths for the sulfur.

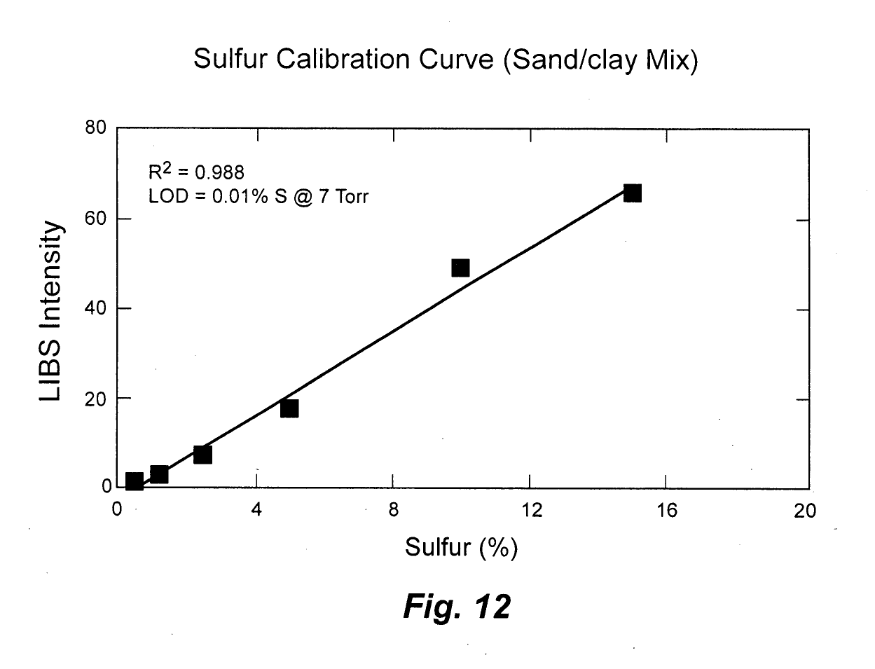

FIG. 13 shows the difference in spectra for a low concentration (2000 ppm) and a high concentration (10,000 ppm) of manganese in a spiked soil sample (synthetic soil/silicate) at a wavelength of about 403 nm.

FIG. 14 shows the difference in spectra for a low concentration (200 ppm), a medium concentration (1000 ppm) and a high concentration (5000 ppm) of zinc in a spiked soil sample (synthetic soil/silicate) at a wavelength of about 213.8 nm.

FIG. 15 shows the difference in spectra for a low concentration (200 ppm), a medium concentration (1000 ppm) and a high concentration (5000 ppm) of copper in a spiked soil sample (synthetic soil/silicate) at a wavelength of about 224.7 nm.

FIG. 16 shows the difference in spectra for a low concentration (200 ppm) and a high concentration (1000 ppm) of chromium in a spiked soil sample (synthetic soil/silicate) at a wavelength of about 425.4 nm.

FIG. 17 shows the difference in spectra for a low concentration (200 ppm), a medium concentration (1000 ppm) and a high concentration (5000 ppm) of lead in a spiked soil sample (synthetic soil/silicate) at a wavelength of about 405.8 nm.

FIG. 18 shows the difference in spectra for a low concentration (2000 ppm) and a high concentration (10,000 ppm) of barium in a spiked soil sample (synthetic soil/silicate) at a wavelength of about 455.4 nm.

FIG. 19 shows the difference in spectra for a low concentration (200 ppm), a medium concentration (1000 ppm) and a high concentration (5000 ppm) of strontium in a spiked soil sample (synthetic soil/silicate) at a wavelength of about 421.5 nm.

FIG. 20 shows the difference in spectra for a low concentration (200 ppm) and a high concentration (1000 ppm) of vanadium in a spiked soil sample (synthetic soil/silicate) at a wavelength of about 438 nm.

FIG. 21 shows a calibration curve for boron in synthetic soil spiked with boric aid with a limit of detection (LOD) of about 200 ppm, the determination at 208.98 nm wavelength for the boron.

### DETAILED DESCRIPTION

The present invention concerns analysis of plants and/or soil for nutrient analysis.

It has now been shown that laser-induced breakdown spectroscopy (LIBS) can be used to accurately measure the concentrations of a set of important plant nutrients and toxic metals in soils or in plant tissues. This invention is a method to accurately measure these elements using a method that is a significant advance over existing technologies by providing an accurate and rapid measurement in a very cost effective manner. The process of the present invention eliminates the need to extract the elements. Target samples generally need only be dried and weighted before introducing them into the LIBS apparatus for measurement. Thus, sample preparation can be reduced to a minimum. A single instrument, the LIBS apparatus, is used to make the measurement for a wide range of elements providing another advance over existing technologies.

A high-energy laser (normally pulsed) is used to vaporize and ionize a small amount of material for analysis. The vaporized material or laser-induced breakdown plasma produces strong optical emission. Spectroscopic analysis of the optical emission gives information about the material being analyzed, such as quantity.

The present method can be used for measuring: primary macro-nutrients such as calcium, potassium and nitrogen; secondary macro-nutrients such as sulfur, phosphorus and magnesium; and micro-nutrients such as chlorine, iron, boron, manganese, zinc, copper, nickel and molybdenum. Analysis for other nutrients by the present process may be readily adopted by those skilled in the art. For each targeted species, a relevant spectral line is identified and potential interferences between spectral lines of other elements must be evaluated. Deployment of a well-calibrated and robust LIBS instrument may provide the large number of accurate measurements needed to rapidly evaluate the nutrient status of soils and plants. In addition, LIBS measurements can be made while in the field and could significantly improve the cost effectiveness of nutrient measurements.

In addition to the various nutrients, the process of the present invention can also analyze plants, e.g., plant material for other elements such as sodium, vanadium, silicon, selenium, barium, strontium and iodine. In such cases, knowledge of the amounts of these materials may be desirable to avoid toxicity levels of such elements. Also, the process of the present invention may be used to analyze for the presence and level of any heavy metals such as iron, lead, arsenic, chromium, cadmium and the like in plants or of similar importance and relevance such levels of heavy metals in any fertilizers and/or soil amendments (e.g., manures) being used.

In analysis of plant samples, the plant material is generally dried to reduce the water content to less than about 5 percent by weight. The drying step is not always necessary, but is generally preferred. Then, the dried material can be ground or pulverized and pressed into a pellet prior to subsequent steps. Again, such pulverizing and pelletizing is only preferred and may be skipped if desired.

In analysis of soil samples, the soil is generally dried to reduce the water content to less than about 5 percent by weight. The drying step is not always necessary, but is generally preferred. As with plant material the soil samples can then be pressed into a pellet prior to subsequent steps.

For analysis in the present process using laser-induced breakdown spectroscopy, the target sample is initially processed and ultimately subjected to the laser light. A Nd:YAG laser (Spectra-Physics Lasers, Mountain View, Calif.) at a selected wavelength of 1064 nm (e.g., 50 mJ pulses of 10 ns) can be focused with a suitable lens with a 50 mm focal length on the targeted sample. Emitted light can be collected though a fused silica fiber optic cable directed towards the plasma from a distance of, e.g., about 50 mm. A spectrograph of 0.5 m focal length can resolve the detected light using a gated-intensified photodiode array detector. For multiple samples, a stepping motor and a movable stage can be coupled to transport the samples through the LIBS instrument and allow collection of spectra from different samples or if desired, from different points of an individual sample. Multiple laser shots can be employed and collected to provide an average at each step. Peak areas can be integrated to yield an estimate of signal intensity for each spectrum and background signal can be subtracted. A typical measurement area for LIBS analysis can be from about 1 to about 5 mm³/pulse.

The present invention is more particularly described in the following example that is intended as illustrative only, since numerous modifications and variations will be apparent to those skilled in the art.

### EXAMPLE 1

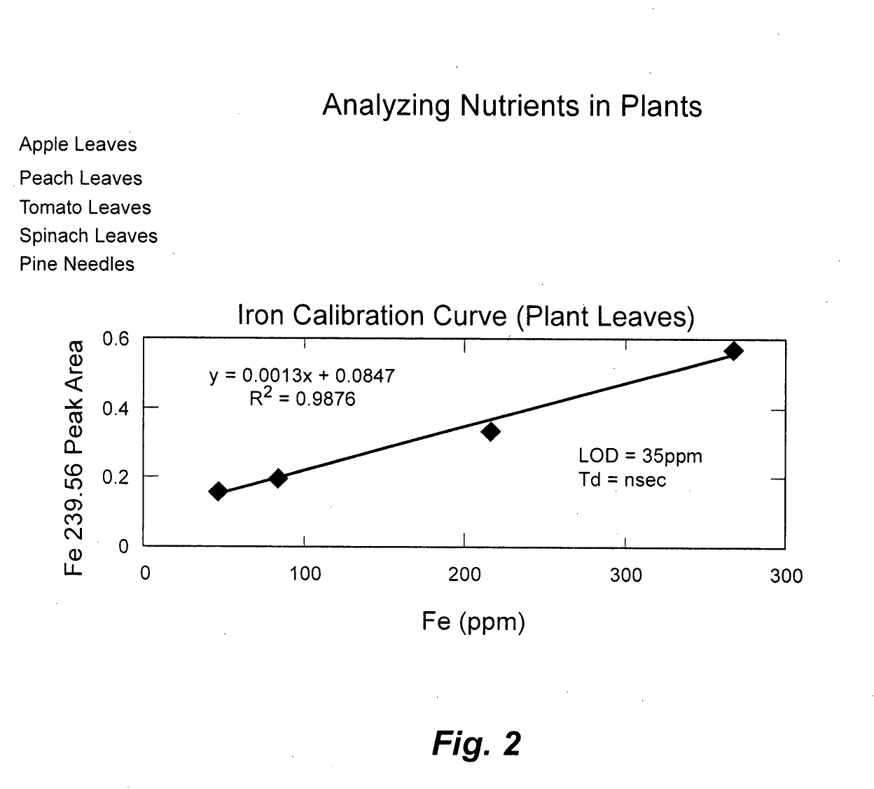

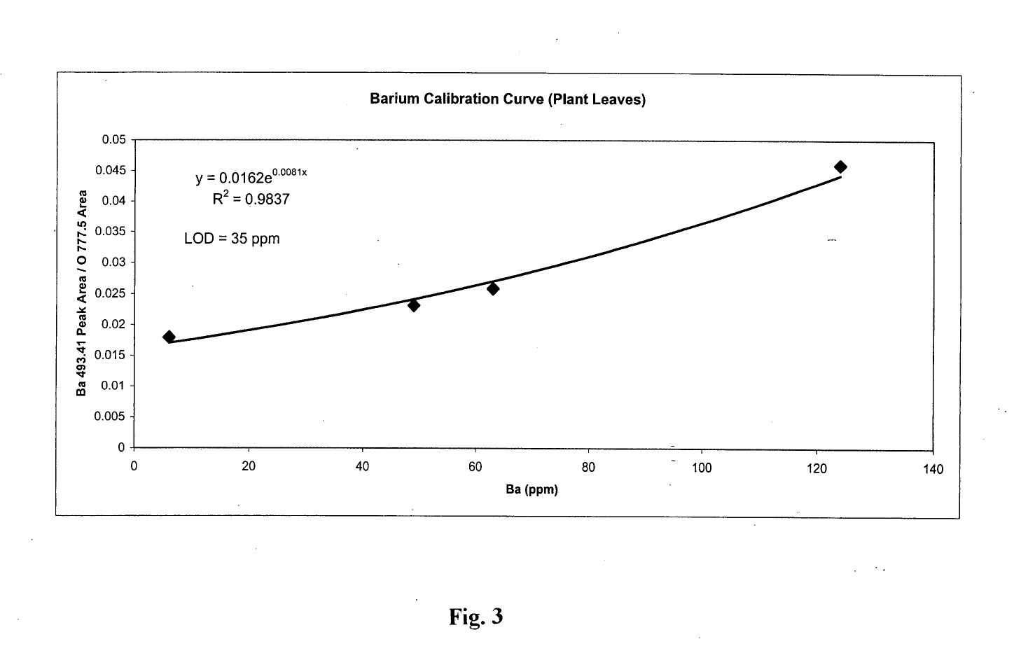

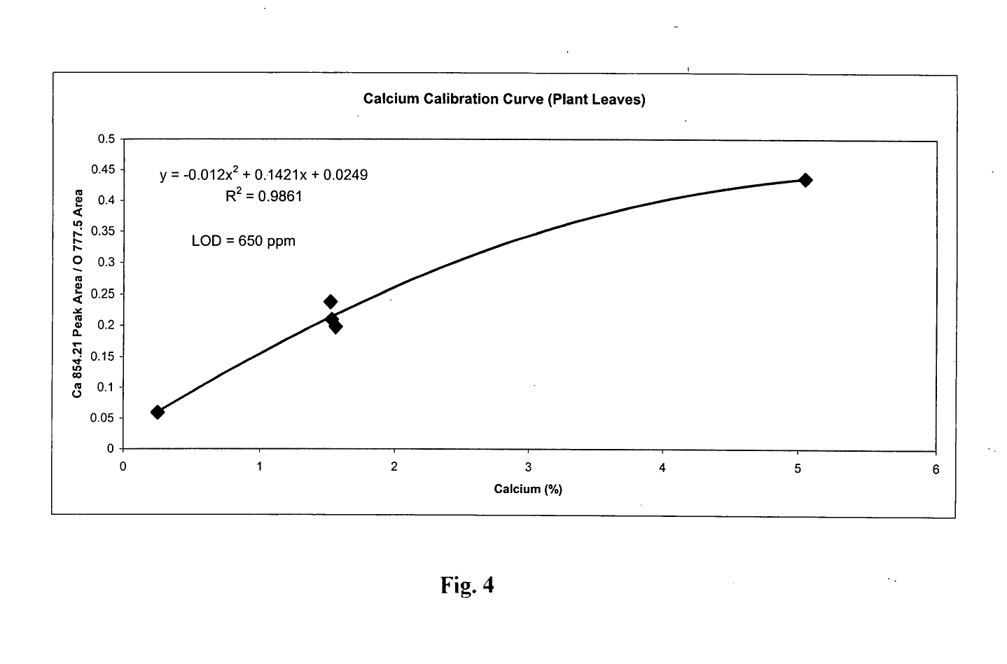

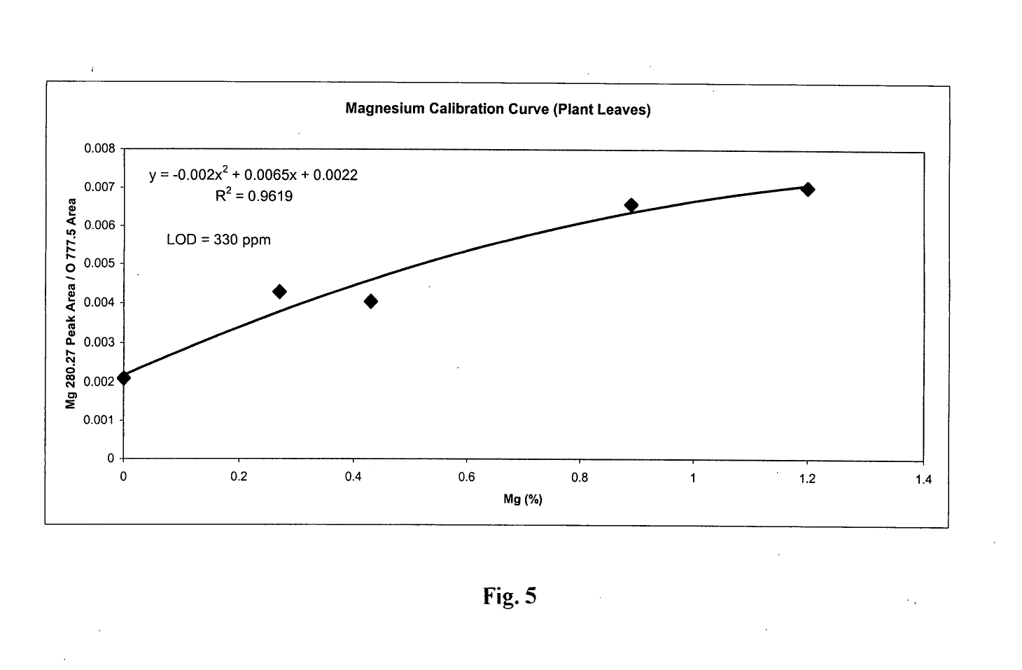

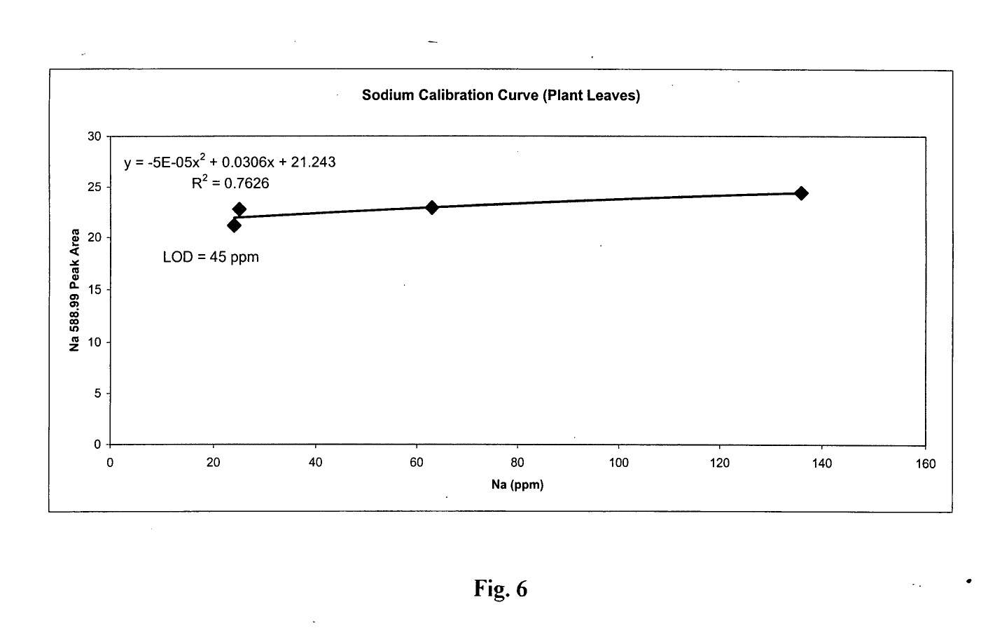

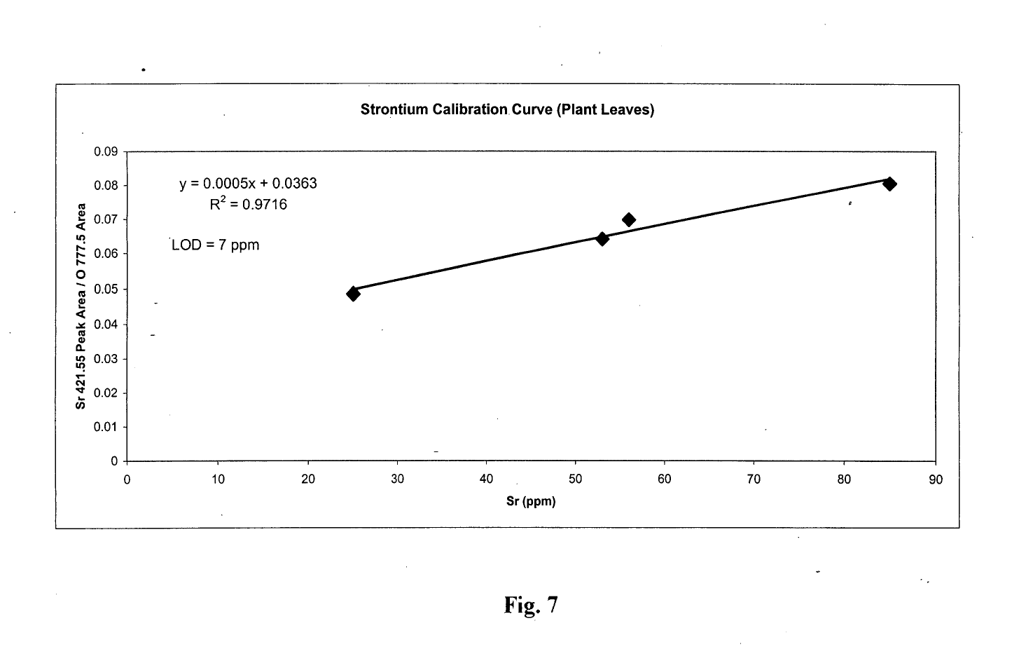

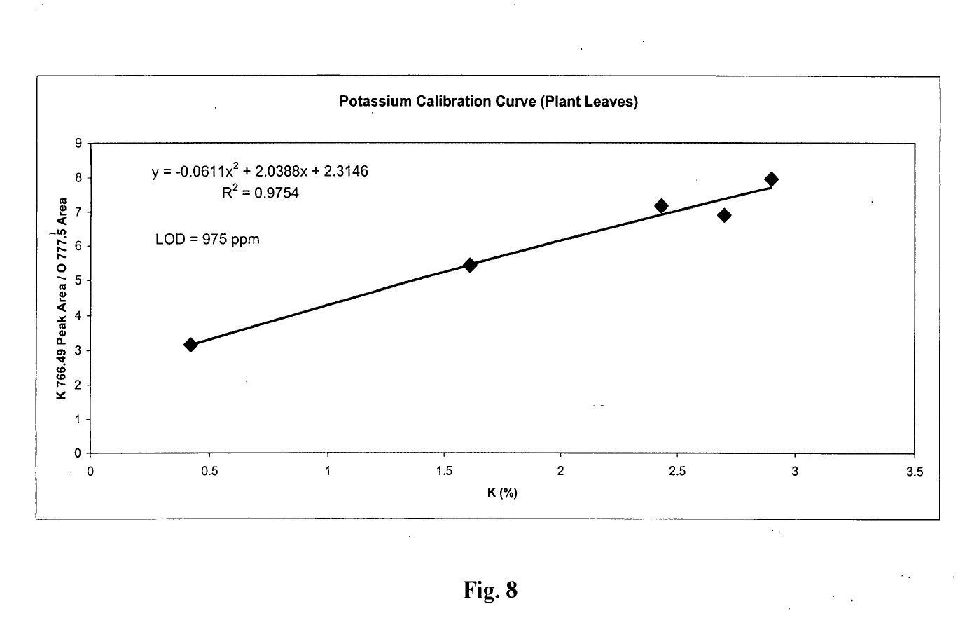

Various plant leaves (apple, peach, tomato, spinach and pine needles) with known levels of targeted species were obtained from the National Institute of Standards and Testing (NIST) standard reference materials, e.g., apple leaves as NIST-SRM 1515, peach leaves as NIST-SRM 1547, tomato leaves as NIST-SRM 1573a, spinach leaves as NIST-SRM 1570a and pine needles as NIST-SRM 1575a. Leaves were measured for calcium, potassium, iron, sodium, strontium and barium using a Spectrolaser 1000HR (XRF Scientific). The particular LIBS instrument generated the necessary bright spark or plasma at the sample, the emission or light from which was subsequently analyzed by a spectrometer and detection system. An argon purge of the container volume containing the sample was carried out to improve sensitivity. Calibration curves were plotted from the standard samples and the particular curve for iron is shown in FIG. 2. Calibration curves were plotted from the standard samples and the particular curve for barium is shown in FIG. 3. Calibration curves were plotted from the standard samples and the particular curve for calcium is shown in FIG. 4. Calibration curves were plotted from the standard samples and the particular curve for magnesium is shown in FIG. 5. Calibration curves were plotted from the standard samples and the particular curve for sodium is shown in FIG. 6. Calibration curves were plotted from the standard samples and the particular curve for strontium is shown in FIG. 7. Calibration curves were plotted from the standard samples and the particular curve for potassium is shown in FIG. 8.

In addition to the listed elements, the detection of other elements from the leaves may be conducted as well.

### EXAMPLE 2

Sample soils were spiked with a general fertilizer (Miracle Gro® All Purpose Plant Food), a lawn fertilizer (Turf Builder® Lawn Fertilizer), or sulfur. The respective soils were then analyzed by first drying and then pressing into a pellet. Subsequently, each pellet sample was measured for potassium, nitrogen or sulfur in the manner of Example 1 except that a more sensitive LIBS instrument was used including a 0.5 m focal length spectrograph (Chromex Imaging Spectrograph, Model 500IS) a gated intensified charge coupled device (ICCD) detector (Oriel, Instaspec V). Also, an argon purge of the sample container volume was used in the measurement of nitrogen levels in the soil to avoid complications from the nitrogen in the air to the measurement level. The emission or light was analyzed by a spectrometer and detection system.

Calibration curves were plotted from the spiked samples and are shown as FIG. 4 (for phosphorus), FIG. 5 (for nitrogen) and FIG. 6 (for sulfur).

### EXAMPLE 3

Sample synthetic silicates (soil-like samples from Bremer Standard Online Catalog, Houston, Tex.), spiked with a general fertilizer (Miracle Gro® All Purpose Plant Food) or a lawn fertilizer (Turf Builder® Lawn Fertilizer), were analyzed in the manner of Example 1 using a Spectrolaser 1000HR (XRF Scientific). The respective soils were then each analyzed by first drying and then pressing the material into a pellet. Subsequently, each pellet sample was measured individually for manganese, zinc, copper, chromium, lead, barium, strontium and vanadium.

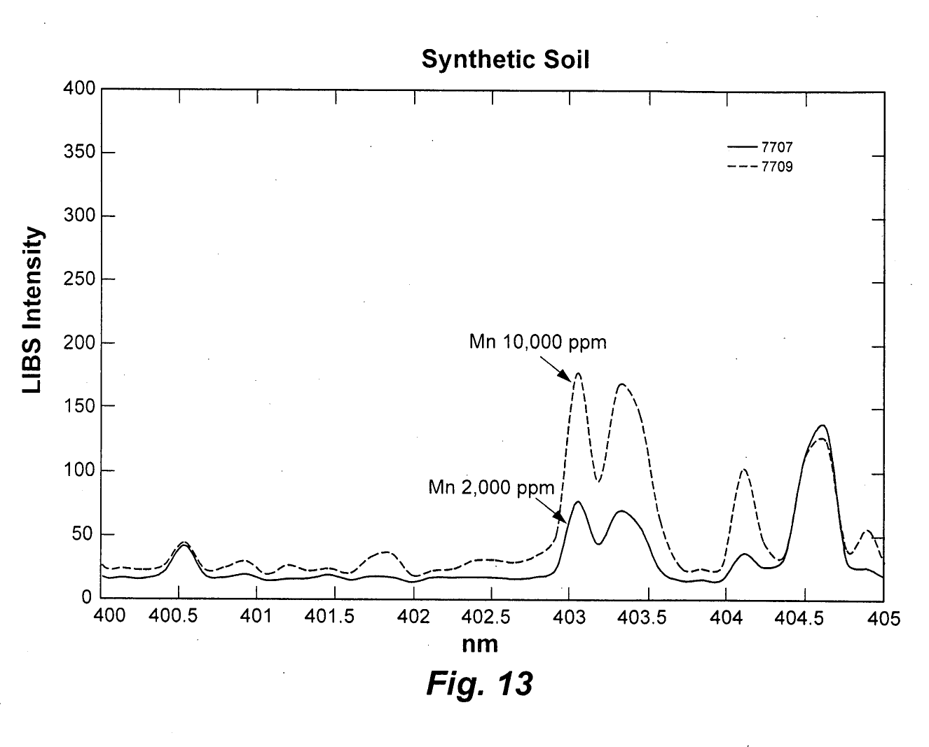

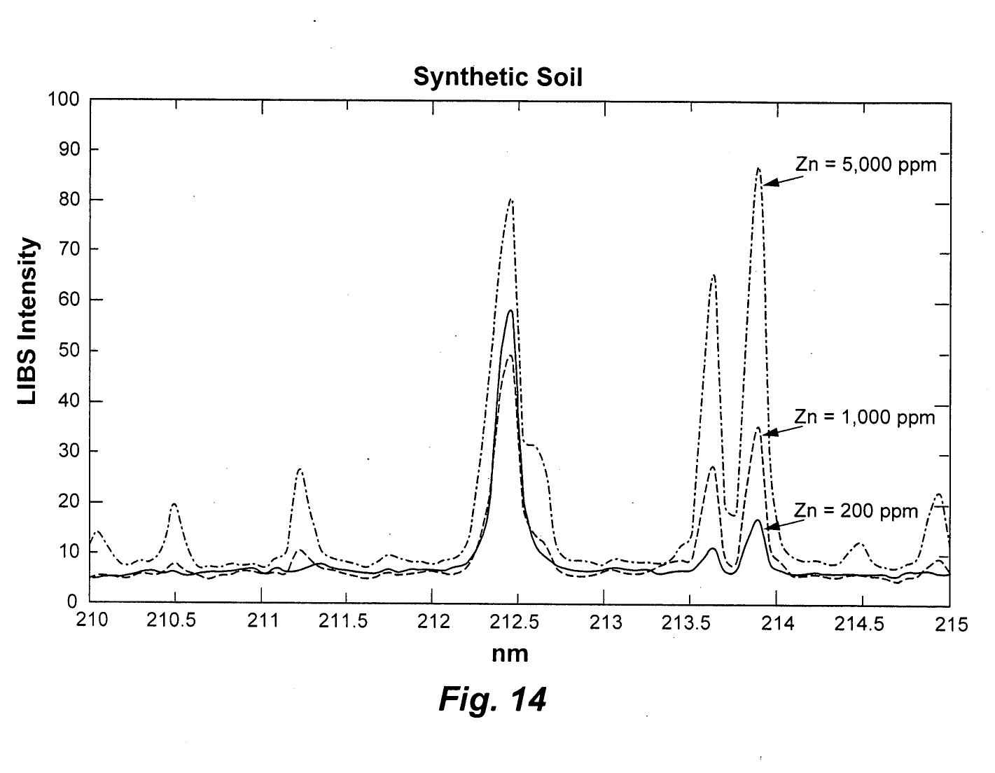

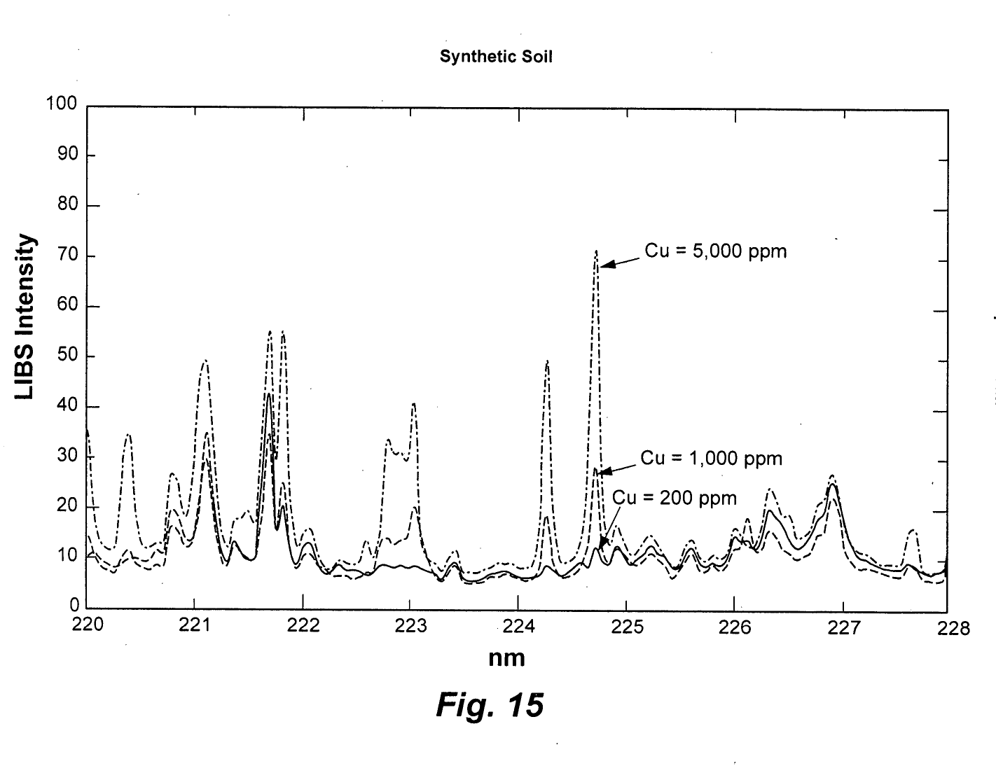

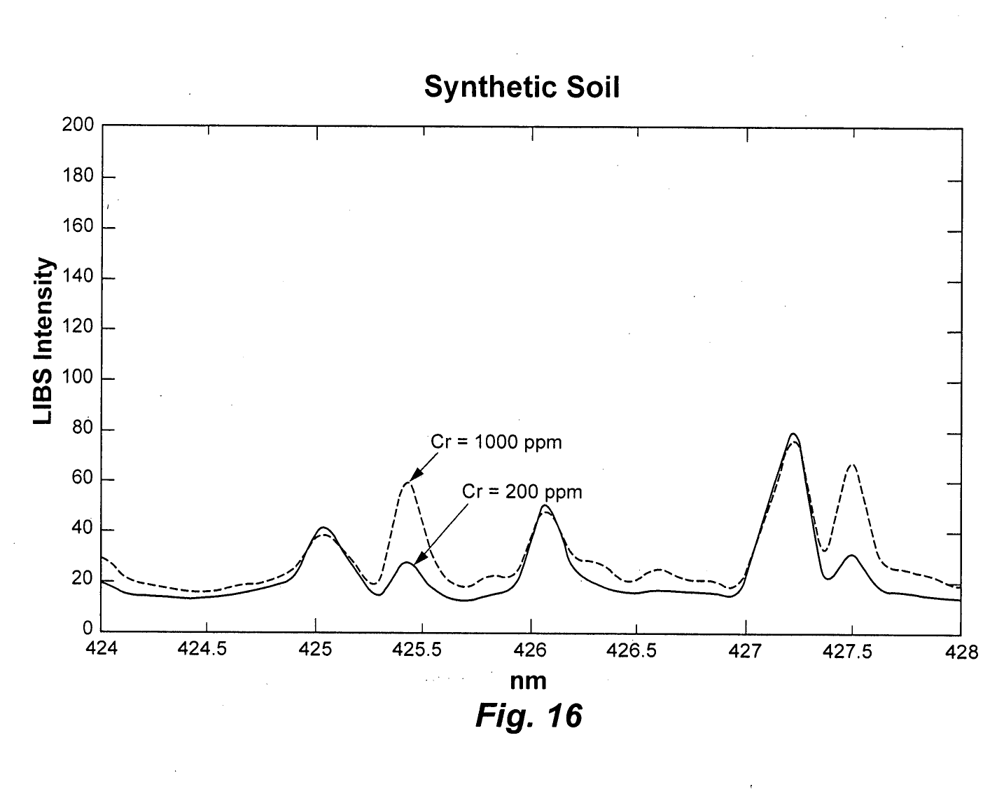

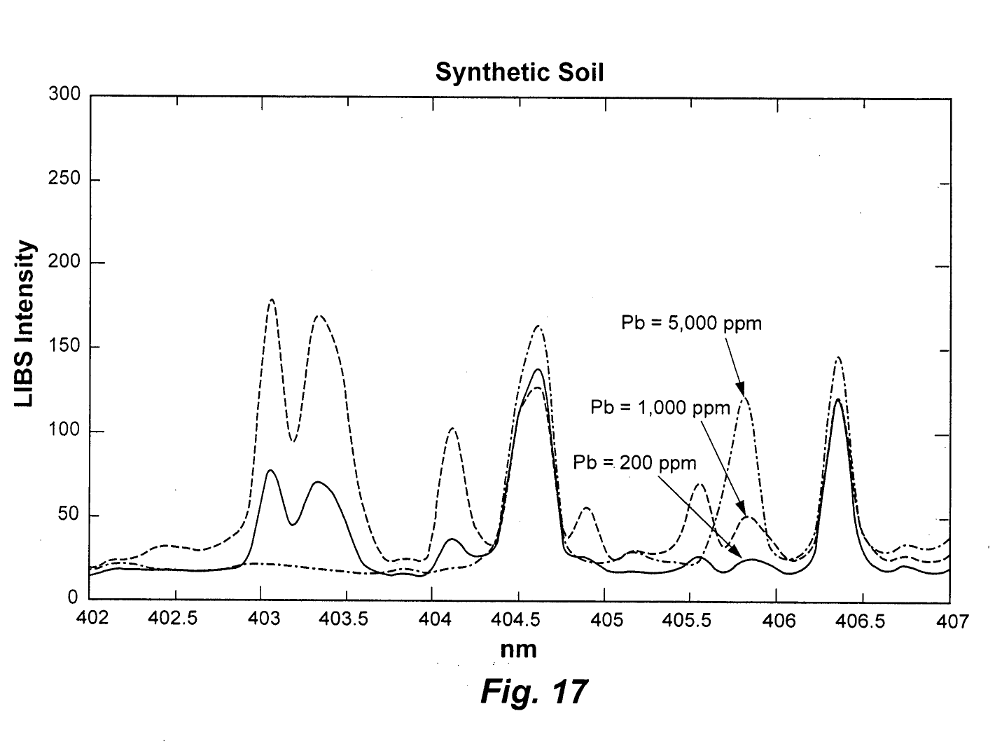

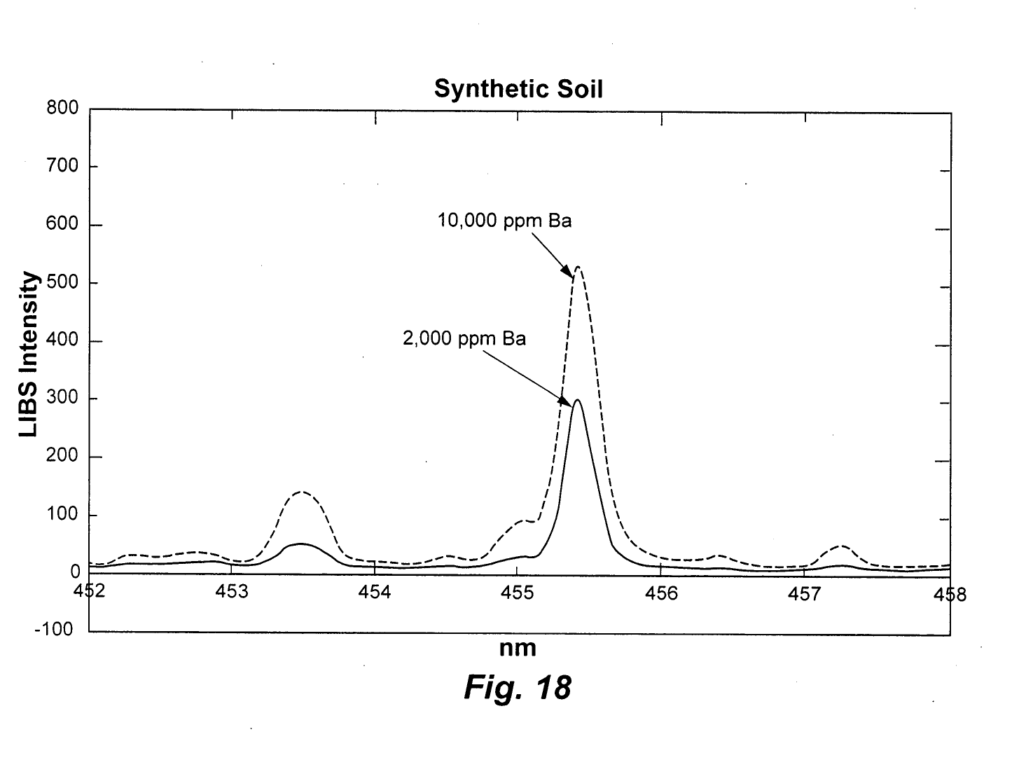

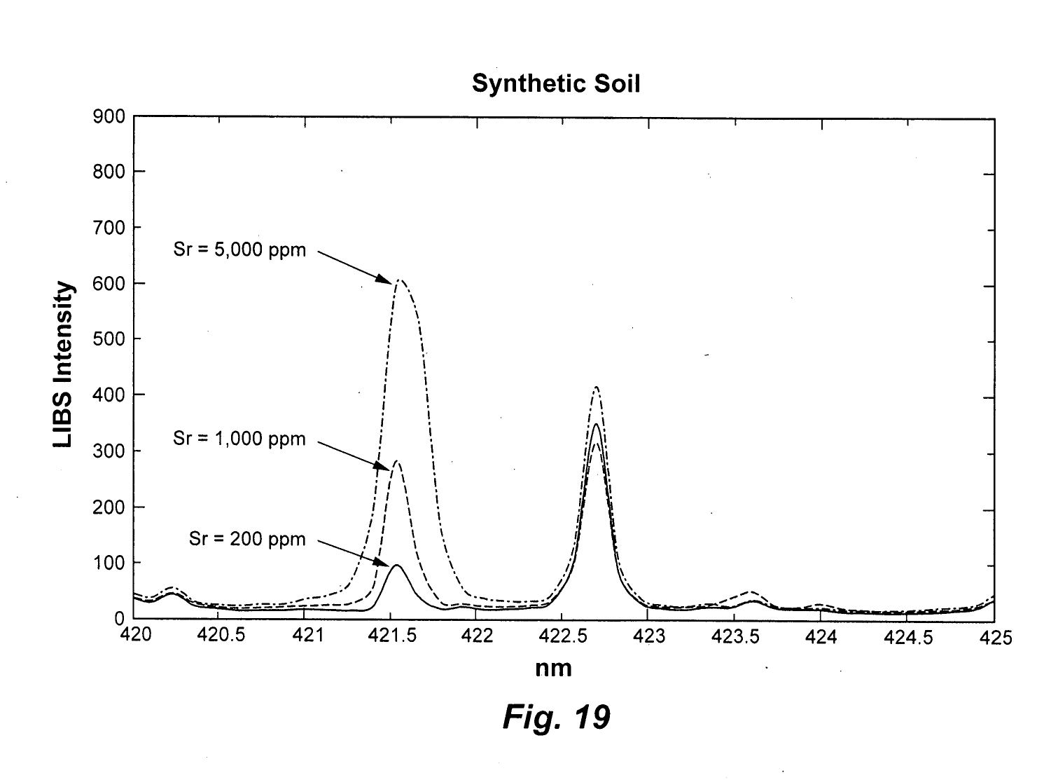

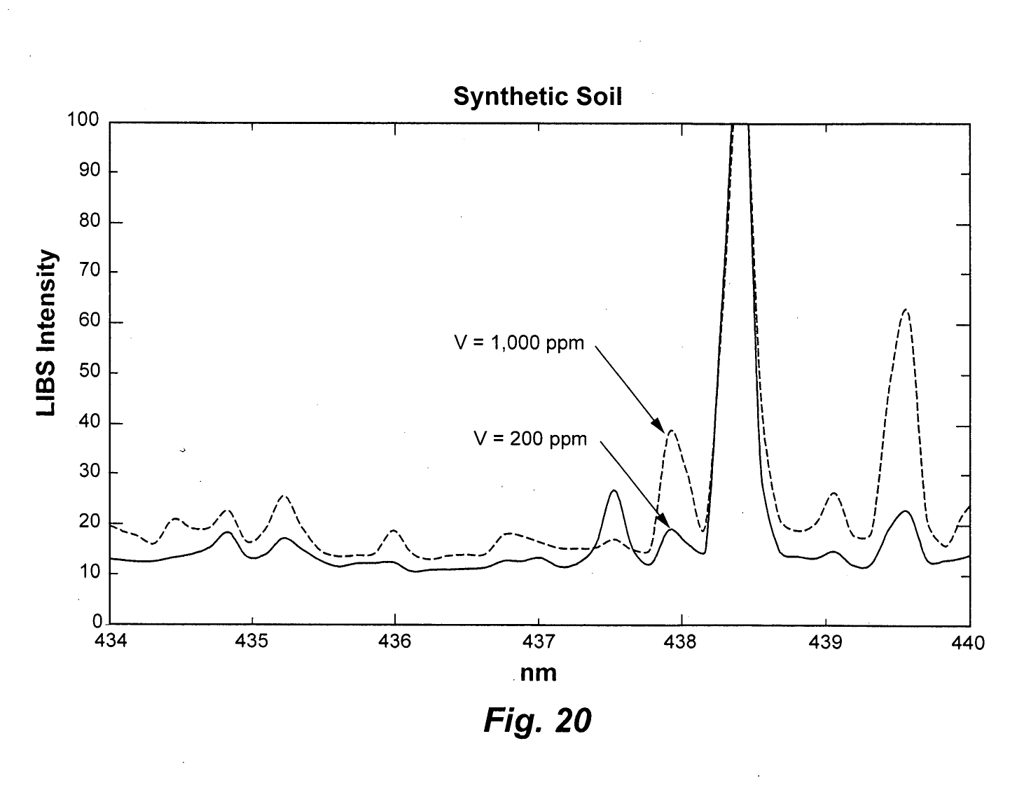

Plots of the spectra were plotted from the spiked samples and are shown as FIG. 13 (for manganese), FIG. 14 (for zinc), FIG. 15 (for copper), FIG. 16 (for chromium), FIG. 17 (for lead), FIG. 18 (for barium), FIG. 19 (for strontium), and FIG. 20 (for vanadium).

### EXAMPLE 4

A sample synthetic silicate (as in Example 3) except spiked with boric acid was analyzed in the manner of Example 1 using a Spectrolaser 1000HR (XRF Scientific). The soil was then each analyzed by first drying and then pressing the material into a pellet. Subsequently, each pellet sample was measured individually for manganese, zinc, copper, chromium, lead, barium, strontium and vanadium.

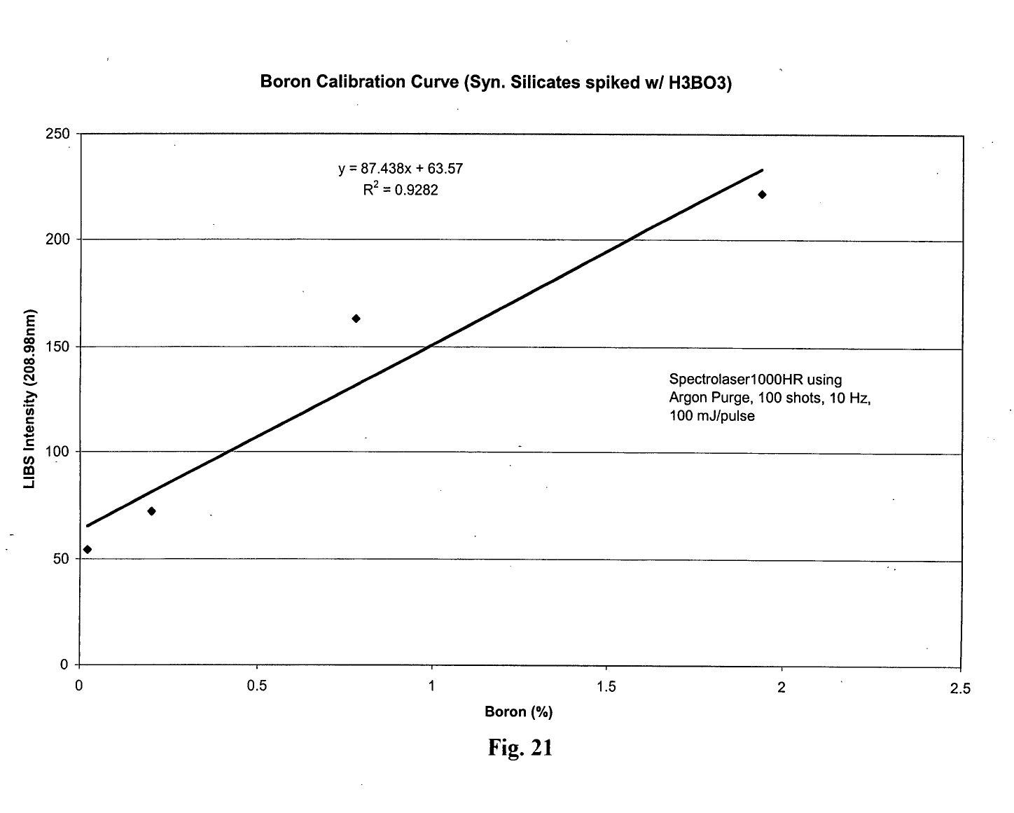

A calibration curve was plotted from the standard sample and the particular curve for boron is shown in FIG. 21. While the lower sensitivity spectrometer allowed the detection of boron in the soil sample, it is expected that the higher sensitivity instrument should be used for measurement of boron levels in plant matter such as leaves where the boron level is typically around 20 ppm.

Although the present invention has been described with reference to specific details, it is not intended that such details should be regarded as limitations upon the scope of the invention, except as and to the extent that they are included in the accompanying claims.

What is claimed is:

1. A process for analyzing the nutrient status of plant matter and/or soil for one or more nutrients selected from among calcium, potassium, nitrogen, sulfur, phosphorus, magnesium, chlorine, iron, boron, manganese, zinc, copper, nickel and molybdenum comprising:
contacting said plant matter and/or soil with a laser source capable of inducing breakdown of the sample whereby an emission from said sample occurs; and,
analyzing said spectral emission for determination of an amount of said one or more nutrients.

2. The process of claim 1 wherein said sample is dried prior to contact with said laser source.

3. The process of claim 1 wherein said laser source is a pulsed laser source.

4. The process of claim 2 wherein said sample is pulverized and pressed after said drying.

5. The process of claim 1 wherein said contact of plant matter and/or soil is under an atmosphere of argon.

6. A process for analyzing plant matter and/or soil for one or more heavy metals selected from among iron, lead, arsenic, chromium, and cadmium comprising:
contacting said plant matter and/or soil with a laser source capable of inducing breakdown of the sample whereby an emission from said sample occurs; and,
analyzing said spectral emission for determination of an amount of said one or more heavy metals.

7. A process for analyzing a fertilizer or soil amendment for one or more heavy metals selected from among iron, lead, arsenic, chromium, and cadmium comprising:
contacting said fertilize or soil amendment with a laser source capable of inducing breakdown of the sample whereby an emission from said sample occurs; and,
analyzing said spectral emission for determination of an amount of said one or more heavy metals.
